(19) United States
(12) Patent Application Publication
(10) Pub. No.: US 2005/0202052 A1
(43) Pub. Date: Sep. 15, 2005

(54) Flavored medicaments to deter or attract and kill microorganisms

(76) Inventor: Clemie M. Lee, Stone Mountain, GA (US)

Correspondence Address: Clemie Lee, 5585 Mountain Crescent, Stone Mountain, GA, 30087, US

(21) Appl. No.: 10/800,824

(22) Filed: Mar. 15, 2004

(51) Int. Cl. A61K 9/00

(52) U.S. Cl. 424/400; 424/439

(57) ABSTRACT

Any orally, injected or topically administered medicament is prepared into a dosage form that is flavored to the pallet of the offending and targeted bacteria or microbe, thereby using the innate flavor preferences of bacteria/microbes to attract them to the pharmaceutical active principle which eradicates them more effectively than non-flavor bonded pharmaceuticals. Further, this invention also addresses the use of flavored medicaments to deter the growth, reproduction, or inhabitation of bacteria or other microbes.

### CROSS-REFERENCE TO RELATED APPLICATIONS

This application claims the benefit of priority of U.S. Patent Disclosure Document 515382 and U.S. provisional patent application Ser. No. 60/411,900, by the named inventor.

### FEDERALLY SPONSORED RESEARCH

Not Applicable

### FIELD OF INVENTION

The present invention relates to pharmaceuticals, and antibacterial solutions enhanced by bonding flavoring to the active ingredients within the medicament that bacteria are naturally attracted to which allures and then kills the offending microbes.

### BACKGROUND OF THE INVENTION

Scientists have known for years that all organisms have a taste preference for their food; man, bird, fish, insects and microorganisms have all exhibited this trait. However, we have never used this knowledge in the manufacture of pharmaceuticals to kill or prevent the growth of microorganisms. This invention relates to the manufacture of pharmaceutical products, and antibacterial compounds where the active ingredient, within the antibiotic, is bonded with flavoring that bacteria are innately attracted to; thereby, accelerating the process of killing the offending bacteria.

Flavor-bonded antibiotics are more effective than traditional non-flavor-bonded antibiotics because the offending bacteria are attracted to the eradicating active ingredient which is bonded to the flavor bait. Due to the increased effectiveness of the flavor-bonded antibiotics or anti-infective, patient wellness is accelerated. Increased antibiotic effectiveness also gives the doctor the option of reducing the strength of the antibiotic's active ingredients; thereby, reducing the risk of side effects for the patient. Further, since bacteria are killed quicker using this invention, the probability of bacterial mutation to form resistance to the antibiotic is also reduced; a most significant benefit for mankind.

### DESCRIPTION OF PRIOR ART

For decades medicine has been flavored to please the pallet of the patient, however, no art exists in this crowded field which utilizes microbe taste preferences to attract and kill them.

Avner Rotman, in U.S. Pat. No. 4,749,575, teaches a method of flavoring medication to the pallet of humans, “which eliminates the unpleasant taste and mouth feel of the medicament”. Rotman's patent, along with several foreign patents also teach methods of flavoring medicaments, to the human pallet, which mask their foul taste: in Europe EP0345787 to John Botzolakis (1985), FR2832635 to Ibanez Fernandez (2003), and in Japan JP5255075 to Yajima Toshihisa (1993); all of these patents addressed varying methods of improving the taste of medication to humans. None of these patents use flavoring to increase the effectiveness of pharmaceuticals against bacteria or other microbes. My invention differs in that the pharmaceutical principle is flavored to the pallet of bacteria; further, none of these patents increase the effectiveness of the medicament as this invention does.

### SUMMARY OF INVENTION

1) Accordingly, it is an object of the present invention to provide antibiotics, anti-infective, and other pharmaceuticals flavored to the pallet of bacteria, rather than medicaments that are not flavored to the pallet of bacteria.

2) It is another object of the present invention to allure the offending microbes via their innate taste preferences and thereby kill them quicker than medicament which is not flavor baited to the pallet of microbes.

3) It is a yet another object of the present invention to expedite patient wellness, due to the increased effectiveness of flavor-bonded pharmaceutical principles.

4) It is a further object of the present invention to reduce potential patient side effects from antibiotic use by reducing the amount of active medicament therein, due to the increased effectiveness of the flavor-bonded antibiotic.

5) It is yet another object of the present invention to reduce the probability of strain resistant bacterial mutation; since flavor-bonded antibacterial compounds eradicate bacteria in less time than non-flavor-bonded antibacterial compounds, bacteria and other microbes have less time to reproduce and birth resistant offspring.

6) It is another object of the present invention to provide flavor-bonded medication to the pallet of microbes, which will attract and kill the targeted microorganisms.

7) It is yet another object of this invention to deter the growth, reproduction, and inhabitation of bacteria and other microbes via flavor-bonded medicament which the targeted microbes innately dislike.

### DESCRIPTION

During the manufacturing process for each anti-infective or antibiotic, artificial or natural flavors are bonded with the active ingredients. Those familiar with existing art are cognizant of the various chemical bonding techniques available to flavor medicament; including covalent bonding and ionic bonding. These bonds may be single, double, or triple in composition. These bonding methods work with nearly all forms of antibiotics, which are far too numerous to list here. However, a brief list of target antibiotics include: Broad Spectrum: tetracyclines & ciprofloxian; Medium-Spectrum: bacitracin, erythromycins, cephalosporins, penicillin, amoxicillin, and streptomycin; and finally Narrow-Spectrum: polymixins. Again this is not a complete list of possible antibiotics/anti-infectives that can be flavor-bonded; instead it is intended to show that any antibiotic can be flavor-bonded whether the microbe is Gram-positive, Gram-negative, or acid-fast.

Flavoring the anti-infective or antibiotic to the pallet of the infecting bacteria is dependent on the target bacteria. Most bacteria prefer a sweet flavor, a taste that denotes energy; others however, prefer a salty taste, yet others have completely different taste preferences. The innate bacterium taste preferences are nature's way of protecting bacteria, bitter taste, in nature, often signaled toxicity; hence some bacterium innately preferred sweet flavors and avoid inhabitation where the food source is bitter tasting. Some plants have a naturally bitter taste, nature's way of protecting the plant from various forms of bacterial infection and wildlife. We can now use this taste-science to protect and heal humans.

The limit of possible flavors and active ingredient combinations which can be used by this invention is as vast as the permutations of bacteria, microbes, suite of possible flavors (sweet, salty, sour, vanilla, bitter, etc.), and possible pharmaceutical medicaments; hence, it is far too vast to state each combination here.

Patients take the medication in the usual manner: oral, topical, or injection. However, the medication is more effective due to its ability to attract the offending bacteria to the eradicating active ingredient, or the medication can deter bacterial infection through its flavor (bitter as an example).

1. Medicament flavored to the pallet of bacteria to allure and kill the offending bacteria: Whereby (a) artificial flavors are bonded to active ingredient(s) in antibiotics or anti-infectives to attract and kill bacteria, or (b) natural flavors are bonded to active ingredient(s) in antibiotics or anti-infectives to attract and kill bacteria, or (c) combination of artificial and natural flavors are bonded to active ingredient(s) to attract and kill bacteria.

2. Flavor-bonded compounds which use flavor preferences to repel microorganisms and prevent or impede their growth and/or reproduction.

3. Flavored-bonded compounds developed to attract and kill microorganisms: including but not limited prokaryotes and eukaryotes.
